(19) United States
(12) Patent Application Publication
(10) Pub. No.: US 2004/0209249 A1
(43) Pub. Date: Oct. 21, 2004

(54) Method of obtaining protein diversity

(75) Inventors: Arnthor Aevarsson, Hveragerdi (IS); Viggo T. Marteinsson, Reykjavik (IS); Gudmundur O. Hreggvidsson, Reykjavik (IS); Jakob K. Kristjansson, Gardabaer (IS); Olafur H. Fridjonsson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Prokaria Ltd.

(21) Appl. No.: 09/878,423

(22) Filed: Jun. 11, 2001

(30) Foreign Application Priority Data
Feb. 23, 2001 (IS) 5863

(51) Int. Cl. C12Q 1/68

(52) U.S. Cl. 435/6

(57) ABSTRACT

The present invention provides a method that can facilitate structure determination of target proteins by x-ray crystallography. It is a method of rational crystallization of members of a target protein family obtained through specific amplification of corresponding genes from natural diversity. The method makes broad biodiversity accessible through sampling and ecological enrichment of diverse high-temperature ecosystems containing thermophilic microorganisms including uncultivable and previously unknown organisms. The method provides means to circumvent many potential problems and bottlenecks in crystal structure determination by selection of suitable proteins directly from nature. The invention combines methods of accessing and screening vast natural diversity and the inherent suitability of thermostable proteins for crystallization in order to maximize probability of successful structure determination.

### RELATED APPLICATION

This application is a continuation-in-part of and claims priority to Iceland Application No. 5863, filed Feb. 23, 2001; the entire teachings of the above application are incorporated herein by reference.

### BACKGROUND OF THE INVENTION

Structural genomics, the large scale determination of three-dimensional structures of biological macromolecules, is expected to have immense impact on biology and medicine. Structural information is mainly obtained by the techniques of x-ray crystallography and has proved to be of greatest importance for understanding protein function as well as for protein design, structure prediction and rational drug design. New ventures in structural biology aim to have an impact on the different steps of the drug discovery process including target discovery and the selection and optimization of lead compounds. The dramatic flood of information and technical improvements in the sequence genomics era are likely to continue in the structural genomics era (1).

Structure determination of biological macromolecules using x-ray crystallography (for general reviews, see 2,3) tends to be time-consuming and prone to failures. Advances in various aspects of this process continue to be made including those being developed for structural genomics projects aiming at truly high-throughput structure determination (see, e.g., 4-6). However, the whole process going from a gene to refined three-dimensional atomic coordinates still has many potential problems and bottlenecks. For example, cloning, expression and purification of proteins is often not without difficulties depending on the properties of the gene and the gene product. Some genes fail to be effectively expressed, proteins from expressed genes can form inclusion bodies and purification of a protein may not produce a pure and monodisperse protein sample. One of the serious bottlenecks in structure determination of proteins using x-ray crystallography is the crystallization step. Many proteins fail to crystallize or produce well diffracting crystals and, even without major difficulties, the whole crystallization process for a particular protein, including the screening and optimization of crystallization conditions, can be very time-consuming. The resulting crystals, although they may be readily obtained and diffract to a high resolution, can reveal many other problems such as difficulties in cryo-cooling, limited lifetime when exposed to x-rays, unsuitable space groups or cell dimensions, high mosaicity and twinning problems. The properties of the protein or the particular crystals may also not lend itself easily to methods of obtaining phase information during structure determination. For single- or multiple isomorphous replacement (SIR, MIR) using heavy atom compounds (see e.g., refs. 1, 7, 8), the crystal may be very sensitive to heavy atom compounds or conversely the protein may not bind a particular metal ion or compound sufficiently as a consequence of a unfavorable proportion or accessibility of certain amino acid residues. Especially the multiple wavelength anomalous diffraction (MAD) method (9), using selenomethionine-substituted proteins, is directly dependent on amino acid composition, i.e., the proportion of Met residues in the protein.

Various aspects of the process of crystal structure determination of biological macromolecules have undergone drastic improvements in recent years. Advances in molecular biology make it possible to produce large amounts of any proteins and pre-formulated and ready-made crystallization screens have simplified crystallization trials. Cryo-techniques and access to synchrotron radiation has greatly improved data collection and new techniques and algorithms, together with increasingly more powerful computers, continue to improve data reduction and phasing. However, the relative ease of a structure determination is still greatly dependent on the physical properties of the protein under study. In turn, these properties are determined by the precise amino acid sequence of the protein. Consequently, it would be highly advantageous to be able to access diverse sources of numerous candidate proteins with slight sequence variations to improve the likelihood of finding a successful candidate for structure determination.

Sometimes, the difficulties, in crystallization or other aspects of the structure determination of a particular protein, have been overcome by switching to the corresponding homologous protein from a different species that proved to be more tractable. Working on homologous proteins from more than one source in parallel has been used as strategy in a class-directed structure determination since one of the proteins will usually be more suitable than others and since the biological information gained can to a large extent be generalized for all the members of a protein family. The increasing number of sequences from genome sequencing projects thus provides better opportunities to avoid problems in structure determination through the use of proteins from the available genes from different sources (10-12). Furthermore, it is well known that proteins from thermophiles have been claimed to crystallize more easily than proteins from mesophiles. Presumably, the crystallizability of proteins from thermophiles is also a consequence of properties that make them thermostable. Consequently, one of the rationales behind high-throughput structure determination in some structural genomics projects is to focus on proteins from a thermophilic microorganism such as Methanococcus janashii or Thermus thermophilus (13-15).

Despite the continuing developments of technical aspects of crystal structure determination, many improvements remain to be made to make it a fast and reliable process and many difficulties can still be encountered. The present invention is intended to improve structure determination by circumventing many of the potential difficulties and problems using methods that provide access to very broad diversity sources of proteins. Even with the significant resources now directed towards genomic sequencing, the total number of organisms sequenced from diverse ecosystems is still very low relative to the total number of organisms in such environments. As less than 1% of naturally occurring microorganisms can be isolated and grown in pure culture, the number of sequenced microorganisms in genomic sequence databases will remain only a fraction of the wild population of species, in a foreseeable future. Therefore, methods to access much broader diversity, than has been obtainable through prior art methods in order to select preferable candidate proteins for structure determination, will be highly appreciated.

### SUMMARY OF THE INVENTION

Many of the potential problems occurring in crystal structure determination are dependent on the properties of the protein under study. The present invention provides methods to access very broad natural diversity, such as in particular thermophilic diversity, and select directly from nature proteins with physical properties suitable for crystal structure determination. The methods described make it possible to overcome the potential limitations of the presently available genes and proteins (e.g., in public databases) by exploration of broad and previously unexplored diversity for a rational selection of candidates for structure determination. This method may make a structure determination possible or may speed up the process by exploring natural diversity and the crystallizability of thermostable proteins.

The underlying rationale and the uniqueness of the invention is the biodiversity-based approach that increases the chances of producing good quality crystals and the success-rate of structure determination. The method is not dependent on the current availability of genes but can generate a large input of genes from different species and in particular thermophilic species, including genes from uncultivable and unknown species. The thermophilic sources of the genes make the corresponding protein relatively well-suited for the purpose and the broad diversity makes further selection of possible by various criteria. The method can be especially useful for the structure determination of a particular protein from more than one species. The invention can make it possible to shift the focus of structure determination from dealing with difficulties in cloning, expression, crystallization, data collection etc. to finding in nature the protein(s) with the properties that makes the whole process relatively easy.

### BRIEF DESCRIPTION OF THE DRAWINGS

The foregoing and other objects, features and advantages of the invention will be apparent from the following more particular description of preferred embodiments of the invention.

FIG. 1 shows phylogenetic relationships of bacterial 16S rRNA sequences as determined by neighbor-joining analysis. The tree demonstrates results obtained by extracting DNA directly from environmental biomass (SRI clones) and by oligotrophic in situ enrichments (OLI clones).

FIG. 2 shows a phylogenetic tree constructed according to the amino acid alignment of the new sequences with sequences of selected amylolytic enzymes from thermophilic bacteria. The tree, constructed with the neighbor-joining method (16) demonstrates varied nature of the amylolytic enzymes in the in situ enrichment cultures.

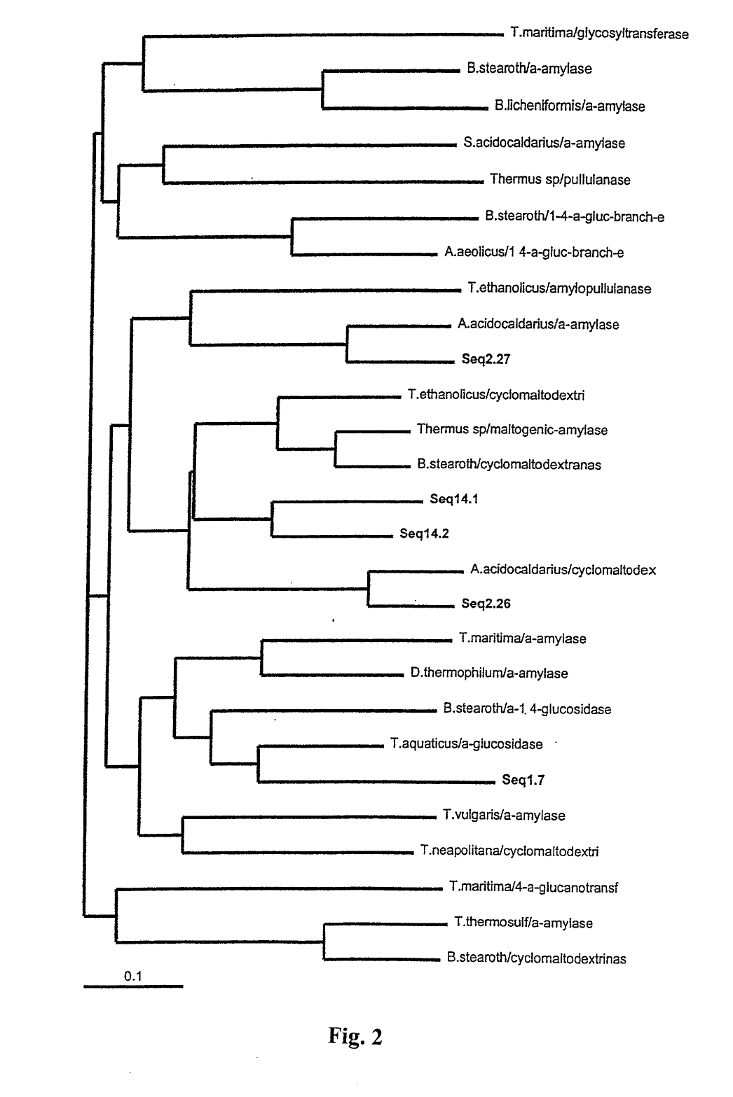

### DETAILED DESCRIPTION OF THE INVENTION

A description of preferred embodiments of the invention follows. In a first aspect, the invention provides a method for obtaining one or more candidate proteins for crystallization from a broad diversity sample, wherein the candidate proteins have desired characteristics to facilitate crystallization, the method comprising: obtaining a broad diversity sample comprising microorganisms potentially having genes coding for one or more proteins having desired characteristics that facilitate crystallization; isolating nucleic acids from the sample; sequencing a plurality of nucleic acid segments comprised in the isolated nucleic acids; selecting from the obtained nucleic acid sequences one or more target sequences based on suitable selection criteria; optionally obtaining from the broad diversity sample one or more additional nucleic acid segments comprising the one or more target sequence or a part thereof, wherein the additional nucleic acid segment codes for the candidate protein or a part thereof; expressing said one or more target sequences and/or additional nucleic acid segments; and isolating the expressed gene product(s) to obtain one or more candidate proteins that have characteristics that facilitate crystallization.

The desired characteristics to facilitate crystallization of the candidate proteins obtainable by the methods of the invention include all features of proteins that will simplify and/or hasten crystallization trials of proteins, and facilitate more efficient crystallization and especially production of crystals suitable for structure determination. Such features include but are not limited to features related to stability, solubility in different solvent systems (both aqueous and organic), tendency of aggregation, protein homogeneity, and more. In particular, as mentioned above, thermostable proteins obtainable from thermophilic organism are generally found to be easier to crystallize, and such proteins are consequently highly preferred as candidate proteins.

In a useful embodiment, the suitable selection criteria comprise one or more criteria selected from the group consisting of the following criteria: a predetermined maximum hydrophobicity of any given region of a predetermined length of the sequence; a predetermined minimum percentage of one or more predetermined amino acid residues; a predetermined maximum percentage of one or more amino acids residues; and combinations thereof. The hydrophobicity criterion may be defined, e.g., such that a target sequence is selected only if does not contain any region of predetermined length—such as about 10 residues or longer, including about 15 residues or longer, such as about 20 residues or longer—that has a hydrophobicity value over a predetermined value according to any given scale for quantifying hydrophobicity, such as the GES-scale (Goldman-Engelman-Steitz hydropathy scale). In a specific embodiment, the hydrophobicity maximum for any given region of a predetermined length is in the range of about −0.8 to about −1 kcal/mole, such as about −0.85 or about −0.90 kcal/mole. As mentioned, a useful selection criterion for the target sequences is a predetermined minimum of one or more amino acid residues. In particular, a minimum ratio of polar amino acid residues may be beneficial for solubility, crystallization and structure determination, such as more than about 4% of a given amino acid, including more than about 3.5%, such as more than about 3%. Such amino acids residues include Asp, Gln, Glu, Asn, His, Lys and combinations thereof A criterion may also be that the target sequence should have a minimum sum of two or more of said amino acids, such as of all said amino acid residues.

Said predetermined maximum percentage of one or more amino acid residues is in a preferable embodment a maximum of the aromatic residues including Phe, Tyr, Trp and combinations thereof, such as less than about 10% of all said residues, including less than about 7.5%, or less than about 6% of said residues.

The features of the candidate proteins that facilitate crystallization will most typically benefit the process of obtaining three-dimensional structural information of the crystallized protein, which is a particularly valuable aspect of the invention.

As mentioned, an important feature of the invention is the use of broad diversity samples. Preferred methods for obtaining such samples are described in detail in the applicants' co-pending application (U.S. patent application Ser. No. 09/770,771, filed 26 Jan. 2001 “Accessing Microbial Diversity by Ecological Methods”); the teachings of which are incorporated by reference herein. Broad diversity samples in this context mean samples comprising or derived from a plurality of species and/or strains of organisms. The samples may be obtained from isolated strains, however, preferably such samples are obtained from natural sources of broad diversity. The samples may be obtained from strains by isolation of the strains from the environment (see, e.g., ref. 17), or from previously isolated strains such as from strain collections such as the American Type Culture Collection (18). Biomass can also be used directly from samples obtained from the environment (see, e.g., 19). In a preferred embodiment of the invention, the broad diversity sample is obtained from a geothermal environment. The broad diversity sample may comprise microorganisms selected from viruses, prokaryotic microorganisms, lower eukaryotic microorganisms, and combinations thereof.

By obtaining broad diversity samples from natural environment, the diversity is not limited by the requirement of cultivation and isolation of strains in the laboratory, where most species fail to grow using currently available methods (20,21). The diversity accessible directly from nature may still be limited by other factors such as the access to diverse ecosystems and by low abundance of certain species and/or the dominance of some species in a specific sample. Several strategies and methods are provided by the invention to increase the accessible biodiversity, for example by sampling several locations representing very diverse environments, preferably such as different high-temperature environments. The diversity of the geothermal sampling environments is expected to be highly correlated to the diversity of the thermophilic organisms obtained.

Particularly preferred embodiments of the current invention involve the use of novel enrichment techniques for enriching the accessible diversity. The enrichment methods alter the composition of the ecosystem before sampling and analysis of the genetic material and enable access to species originally found as minor fraction of the total population. Such enrichment methods comprise obtaining a sample containing microorganisms from an environment in which they naturally occur, maintaining the sample under conditions substantially similar to the environment from which the sample was obtained for expanding the microbial population, and allowing a sufficient quantity of a microbial population to expand. The enriched microorganisms may include viruses, prokaryotic microorganisms, such as belonging to Bacteria and Archaea, and lower eukaryotic microorganisms such as fungi, some algae and protozoa. The microorganisms may be cultured or uncultured microorganisms and such microorganisms may be extremophiles, such as thermophiles and psychrophiles, etc. Sources of microorganisms as a starting material would be from different natural environments including oceans and lakes, and particularly from extreme environments such as terrestrial and marine geothermal areas. As used herein, “enrichment” is intended to mean the act of increasing the proportion of the desirable organism by introducing nutrients and conditions or solid support required for increasing the population of the organism of interest in their natural environments thereby taking advantage of natural fluctuations influencing species richness. As used herein, “culturing” is intended to mean growing microorganisms on or in a controlled or defined medium. “Expanding” cell populations is intended herein to mean culturing cells for a time and under conditions that allow the cells not only to grow and thrive, but to multiply to obtain a greater number of cells at the end of the expansion than at the beginning of the expansion. Through the methods of enrichment, culturing and cell population expansion, a sufficient quantity of nucleic acids can be obtained for further study and/or isolation. The methods involve the use of natural fluids as base for media and various conditions for preferably inducing growth of groups of microorganisms with genes encoding desired biological catalysts or that produce bioactive small molecules. The natural fluid can be from an oligotrophic environment or it can be synthetically replicated in the laboratory to mimic a natural environment. As used herein, “oligotrophic” is intended to mean an environment characterized by a low accumulation of dissolved nutrients and organic components for growth of microorganisms.

In useful embodiments of the method, liquid from the environment (e.g., hot spring fluid) is collected into culture containers. The culture containers may be made of synthetic or other material that may be permeable for small molecules and gases and contain various culture volumes. Temperature, pH and/or conductivity probes that record the data at some time intervals for short or long period, and some artificial support for colonization may be inserted in the container. The containers may be placed in an in situ environment (such as in a hot spring) at various temperatures and depth or they may be incubated at specific conditions such as with programmed fluctuations in the laboratory. The containers may be filled with natural liquid and different gases (e.g., nitrogen, hydrogen) in various volumes as headspace of the enrichments. Various substrates in low concentration, from complex nutrients (e.g., yeast extract) to monomers (e.g., amino acids) may be added to the culture containers as well as other vital increments at will. In order to induce growth of microbes that contain genes coding for desired enzymes such as amylases and that may be active at certain temperature range, a container may be placed in a hot spring with in situ geothermal fluid and starch or other appropriate substrate, nutrients or inhibitors. Also, a probe for continuous monitoring of the temperature or pH may be put inside the containers. The additions can also include carbohydrates (e.g., cyclic sugars, monosaccharides, disaccharides, oligosaccharides, polysaccharides, glycoproteins, lectines and phosphate esters of carbohydrates), proteins (e.g., peptides, polypeptides, polypeptone, keratins, collagen, elastin etc.), fatty acids (e.g., propionate, butyrate, succinate, long chain fatty acids etc.), nucleic acids (e.g., nucleosides, nucleotides, deoxyribonucleic acids, ribonucleic acid etc.), lipids (e.g., triacylglycerols, phosphoglycerides etc.), or various other organic compounds such as alcohols, oils, cell extracts, dietary fibers, etc. Also, other modulating compounds like inhibitors (e.g., heavy metals, organic solvents or detergents) and anti-microbial agents (e.g., drugs, antibiotics and preservatives) may be added. Various modes of energy conservation, other than organic substrates may also be used, such as hydrogen or sulfur compounds as electron donors and carbon dioxide, oxygen, nitrate or sulfur compounds as electron acceptors. A small sample of natural biomass typically milliliters of liquid, milligrams of solids or any dilution thereof may be used as additional inoculants.

The containers may be placed for incubation at the same location where the fluid was taken or it may be incubated at a different place such as a laboratory. Cell growth may be easily monitored by phase-contrast microscopy and the enrichment can be terminated at any time at any cell density. Series of enrichments can be done in different containers containing fluid from the same site with different incremental additions. After monitoring the cultures, the cells can be mixed in different proportions before concentrating the cells by centrifugation, in order to normalize the genome representation before DNA is extracted, followed by isolation of nucleic acid segments such as by PCR amplification, or making of gene libraries. As used herein, “normalized” refers to making the amount of cells of different species approximately equal in quantity or numbers before DNA extraction of cell mixture in order to obtain a more even representation of their genomes.

The enrichment methods described herein offer the ability to recover high diversity of active cells that have been growing under known and controlled physiological states during enrichments. Another advantage is that nucleic acid samples are more easily isolated and purified with previously described culture techniques than, from “dirty” environmental samples. Furthermore, large amounts of un-fragmented DNA may be obtained which is free from enzyme inhibitors and there is less risk of undesirable artificial PCR amplifications. Also, these methods allow complete sequencing of whole genes, of gene operons or clusters of genes, for example genes that code for enzymes for a particular biosynthetic pathway (e.g., metabolism of (synthesis and/or degradation) amino acids, vitamins, coenzymes or other secondary metabolites such as antibiotics and pigments). Conditions of the enrichments may be influenced by chemical additions to induce growth and allow selective target groups of microbes to flourish. The target groups of the microbes are influenced by the chemical additive. For example, one may enrich for microorganisms that use starch in their metabolism and contain genes encoding for desired biological catalysts, e.g., amylolytic enzymes that are active at least at 65° C. The fluid in the container is supplemented with starch for inducing growth of such microorganisms which are able to use starch as an energy source. The container containing the microorganisms and inducer is placed at some depth in a hot spring at a desired temperature. After time the culture is collected and the data from the temperature probe is read to record the actual temperature fluctuations during the enrichment period. Allowing the microbes to grow in the presence of starch would enrich for organisms able to induce starch degrading enzymes. DNA may be isolated and the culture screened for microbial diversity and/or diversity of genes encoding amylolitic enzymes. Various substrates in low or high concentration may be added such as but not limited to carbohydrates (e.g., cyclic sugars, monosaccharides, disaccharides, oligosaccharides, polysaccharides, glycoproteins, lectines and phosphate esters of carbohydrates), proteins (e.g., peptides, polypeptides, polypeptone, keratins, collagen, elastin etc.), fatty acids (e.g., propionate, butyrate, succinate, long chain fatty acids etc.), nucleic acids (e.g., nucleosides, nucleotides, deoxyribonucleic acids, ribonucleic acid etc.), lipids (e.g., triacylglycerols, phosphoglycerides etc.), or various other organic compounds such as alcohols, oils, cell extracts, dietary fibers, etc. Also other modulating compounds can be used such as but not limited to inhibitors (e.g., heavy metals, organic solvents or detergents) and anti-microbial agents (e.g., drugs, antibiotics and preservatives). Various modes of energy conservation other than organic substrates may also be used, such as hydrogen or sulfur compounds as electron donors and carbon dioxide, oxygen or sulfur compounds as electron acceptors. Environmental sampling and enrichment of preferred geothermal species can be further rationalized and targeted through the compilation and use of a specific database such as a database containing geographic, physical, chemical and ecological information on various geothermal and individual hot springs.

DNA can be prepared from strains using standard methods (22) and from biomass in environmental/enrichment samples with methods which may depend on the type of the sample, e.g., a relatively clean water sample or a sample containing high concentration of particles from sand or mud (23,24). When extracting DNA directly from an environmental sample, such as hot springs, many physical, chemical and biological factors can interfere with the extraction or with the nucleic acid. DNA isolation is an important and difficult step in the generation of a broad diversity DNA library from an environmental sample, but no reliable method exist which can deal with all the interfering barriers found in an environment. Preferably, cells may be separated, cultured and harvested from interfering factors in the environment by using the enrichment techniques described herein.

The plurality of nucleic acid segments which are sequenced are preferably obtained by PCR-based amplification methods but may also be obtained by other methods, many of which are known in the state of the art. In the case of PCR-based amplification-selection, primers used can be designed, on the basis of sequences from a protein family of interest, to obtain a plurality of nucleic acid segments comprising nucleic acid segments suspected of coding for a protein or part of a protein from said protein family. The term “protein family” in this context is to be understood as comprising proteins that share sequence, structural, or functional characteristics, such as sequence similarity, conserved sequence motifs, structural domains, structural folds, or functionalities such as active sites including binding sites. Preferably, such shared characteristics are reflected in the genes encoding the family proteins, such that proteins family members may be found and selected by genetic screening methods as described herein. Specific gene fragments can be amplified from the isolated DNA using amplification methods such as the polymerase chain reaction (25-30).

Amplification of nucleic acid segments according to the invention is dependent on the specificity of the primers which can be very variable depending on the design and the underlying conservation of regions complementary to the primers. The use of relatively unspecific primers can lead to the amplification of sequences not belonging to the genes being targeted.

In one preferred embodiment of the invention, the step of isolating nucleic acids comprises amplifying the copy number of genes by the use of primers that are designed on the basis of alignments of sequences from specific protein families after alignments of sequences from gene families. The primers used are designed on the basis of conserved regions in these families and include techniques of using both two degenerate, forward and reverse primers or only a single degenerate primer where the second primer is targeted to an adapter site or one supplied by a cloning vector (31-33).

Primers for use according to the invention may further be designed to preferentially screen and amplify candidate sequences from the protein family of interest that have one or more selected features. In useful embodiments PCR primers are designed to selectively amplify only those members of a gene family in natural diversity that have the desirable properties. An example is the design of primers for selective amplification of genes closely related to a specific member or subgroup of a family or only genes with specific structural features in the corresponding protein such as conserved binding site features. Similarly, the enrichment techniques provided herein may suitably be used to enrich species with desirable properties in the natural population being sampled, such as e.g., the enrichment of species being able to utilize a certain substrate and which are then likely to possess a certain enzyme activity corresponding to a specific gene family. The plurality of DNA nucleic acid segments may also be selected more or less non-specifically, e.g., to obtain a library of diverse sequences from which target sequences can be selected based on suitable selection criteria.

To use proteins from thermophiles or other sources in order to obtain structural information relating to a protein family, the target protein family has to exist in a microorganism being sampled. The thermophilic sources are known to be found within the kingdoms of Bacteria and Archaea. The probable presence and spread of a specific target protein family among thermophiles may be seen through analysis of publicly available sequences. Conservation of specific protein families across species and kingdoms can be found through sequence comparison such as by using the algorithm implemented in the BLAST program((http://www.nbci.nlm.nih.gov/BLAST; (34) or by using precompiled databases such as Pfam (http://www.sanger.ac.uk/Pfam; (35) and COG (Clusters of orthologous groups, http://www.ncbi.nlm.nih.gov/COG; (36)).

The amplification of genetic material from samples of biomass is based on PCR primers that should be specific for the selected gene family. Alignment of sequences can be done using alignment programs such as ClustalW (37) for the visual identification of conserved regions. The design of the primers can be done with the CODEHOP method (Consensus Degenerate Hybrid Oligonucleotide Primers, (38) which requires that a number of sequences of members in the family are available with conserved regions containing at least 3 or 4 highly conserved residues and adjacent moderately conserved regions.

The amplified sequences are sequenced with suitable standard methods such as the dideoxy chain termination method equipment (39) using the appropriate equipment and the resulting sequences stored on digital media. The sequences may thus be identified by sequence similarity to known sequences through comparison with sequence databanks for example with search programs such as BLAST (34). Sequences belonging to the targeted gene family can successively be added to a pool of sequences of members of the family and compared by alignment using programs such as ClustalW (37).

The suitable selection criteria to select target sequences include sequence-homology based criteria wherein target sequences are selected that are related to sequences of protein families of interest.

Various selection criteria can further be used for the selection of suitable candidates from the plurality of sequenced nucleic acids. Such embodiments include but are not limited to: i) Sequence variability of selected candidates may be chosen to represent different subgroups within a family and spread variability in sequence space in order to spread physical properties; ii) Selection of candidates can be made with respect to their similarity to a certain sequence. Selected candidates may be for example those most similar to a given sequence such as the sequence of a human member of the protein family; iii) Certain observed trends concerning properties of proteins suitable for structure determination, from retrospective analysis of biophysical data, can be used for the screening of the sequence library to select promising candidates. In an analysis of data from high-throughput structural genomics project (40), data mining methods (in particular decision trees) were used for analysis and development of prediction rules. It was found for example that proteins likely to be insoluble have a hydrophobic stretch longer than 20 amino acid residues (average GES-scale hydrophobicity (Goldman-Engelman-Steitz hydropathy scale) less than −0.85 kcal/mole), proportion of Gln residues proportion of aromatic residues more than 7.5%. Similarly, prediction rules have been generated for crystallizability and expressibility of proteins from these results (see http://bioinfo.mbb.yale.edu/labdb/datamine) which indicate for example correlation between the proportion of Asn residues and crystallizability; iv) Candidates with a suitable frequency or desired number of certain amino acids can be selected that benefit structure determination, in particular to facilitate phasing methods. In one useful embodiment, target sequences are selected wherein the proportion of methionine residues is suitable for multi-wavelength anomalous diffraction, such as in the range of about 1 methionine per 70-80 amino acids. Other amino acids residues, such as e.g., Cys residues, may be useful if conveniently located in the folded protein to bind heavy atoms for use in isomporphous replacement methods. It may be desirable to limit the number of potential binding sites for some heavy atom compounds by for example having only one Cys residue in a candidate protein.

In highly preferred embodiments of the invention, the candidate proteins for crystallization are intended for obtaining crystal structure information. However several other uses of crystallized proteins are contemplated, such as for immobilizing proteins with desired functionalities, e.g., immobilized enzymes for biotransformation processes (41), that may be obtained with the current invention. Crystallization may also be used as a purification step of a desired protein.

The candidate proteins of the invention can be utilized to provide valuable structural information for a selected gene families. Three-dimensional structure is much better conserved than amino acid sequence. Structural deviation of homologous proteins measured by structural superposition is very limited compared to their sequence deviation (42). Structural information from one member of a protein family can to a large extent be extended to other homologous members of the same family even across well-separated phylogenetic domains. Comparison of structures of homologous proteins from thermophiles and non-thermophiles has revealed a high degree of structural conservation, especially in the active site. The adaptation of proteins to various physiological temperatures does not generally require drastic structural modifications and relatively subtle differences are usually found between thermostable and more thermolabile protein (43, 44). Crystal structures of proteins and other macromolecules from thermophilic microorganisms can provide very valuable structural information with potential use in various fields including protein design, proteomics, structural genomics, antibiotic design and other structure-based drug design for human drug targets. The following embodiments demonstrate the value of the proteins and information obtained by the current invention.

In useful embodiments the candidate protein comprises an active site of a protein family, wherein the term active site is meant to include binding sites both for another protein molecule or a small molecule or other biomolecule such as e.g., nucleic acids.

Many of the commercial enzymes presently in use, both high bulk industrial enzymes such as a-amylases and specialty enzymes such as DNA polymerase, are from thermophilic bacteria and other bacterial sources. Structural information on these enzymes obtained directly or indirectly from homologous proteins obtained by the invention through homology modeling, can be used for protein design in order to alter properties such as substrate specificity, solubility and thermostability.

The plurality of obtained sequences of a selected protein family may be useful in demarcating regions of conservation and variability. It can also be helpful for elucidating structural determinants of active sites or other important functional properties such as thermostability or tolerance to adverse conditions. Such determinants include both single amino acid residues or larger regions that can serve as targets for rational modifications. The determinants also allow a focused approach to directed enzyme evolution using a variety of techniques such as DNA-shuffling, staggered PCR or the construction of chimeraic genes, whereby variability is generated either by mutagenesis or by using the variability in the sequences obtained.

In a certain embodiment, the protein family of the candidate protein comprises a protein in a pathogenic organism. A large number of the proteins of pathogenic bacteria, viruses and parasites will have corresponding protein family members in thermophilic organisms, thus representatives of said families are likely to be found with the methods of the invention.

Another example of the potential utility of the invention is for the crystallization of specific potential drug targets and subsequent 3-dimensional structure determination to be used for rational structure-based drug design to produce new antibiotics. In this case, the protein being crystallized could be a candidate protein from a thermophile homologous to the actual drug target in the pathogen. This could be useful in cases where appropriate target in a pathogen fails to crystallize or presents other difficulties in structure determination. It could also be very useful for the design of broad-spectrum antibiotics which may also be effective against a target in a thermophilic bacteria as well as a target in a pathogen. The structure of the protein in the thermophile could thus be directly used for the structure-based drug design and/or provide a homology-model of the target in a pathogen. Design of broad-spectrum antibiotics might also benefit from the availability of structures of a specific target from a number of bacterial species. The structure of one member of a protein family can also facilitate structure determination of other homologous members through the technique of molecular replacement.

The whole-genome sequencing projects have sparked many other high-throughput biological projects such as proteomics and structural genomics projects. Assignment of function to a certain gene product can greatly benefit from knowledge of the three-dimensional structure of a particular protein and in most cases even from the structure of a homologous protein. The aim of some of the structural genomics projects is to determine structure of any member of a selected protein family to aid assignment of function and homology (12,5). These efforts can potentially benefit much from the use of proteins that are obtained by the current invention.

Another example of utility of this method is the crystallization of a (thermostable) bacterial homologue of a human protein (or of another eukaryote). The structure of the bacterial protein is likely to have the same general structure in 3-dimensions and the active site may be well conserved. The structural information gained from the bacterial protein may thus be used to aid research on the human protein in several ways:

a) Determination of function. In some cases, the function of a protein of interest, such as a protein found to be linked to a certain disease, may be unknown. Knowledge of the structure of a protein has been shown in many cases to help identifying the function of the protein. The bacterial homologue will have the same fold as the human protein and structural comparison may be used to identify structural relationship to other proteins with known structure and function. A similar function can often be inferred from those structural relationships. The structural determination can itself also reveal cofactors, metal ions or other ligands bound to the protein which may indicate the possible function of the protein which may be verified experimentally (45-47,40,14)

b) Predicting the effects of mutations. A certain human protein may have known mutations which e.g., are known to be linked to a human disease. Structural information can be invaluable in understanding the effects of mutations and give profound insight into the molecular basis of a disease caused by the mutation and suggest routes to the design of drugs against the disease (48).

c) Predicting protein-protein or protein-ligand interactions. The structural information can give clues to the location of surfaces involved in interaction with a small-molecule ligand or another protein. The structure may allow these interactions to be modeled through docking experiments.

d) Facilitate structure determination. The structure of a bacterial protein can be efficiently used to facilitate structure determination of the homologous human protein. The bacterial protein can provide a search model that may be used for molecular replacement which is often a much more convenient and more rapid method for structure determinations than other more elaborate methods such as isomorphous replacement or multi-wavelength anomalous diffraction.

e) Structure-based (rational) drug design. Structural information can be used in a rational way for the design of a drug which can be e.g., an inhibitor of the human protein (49-51). The structure of the bacterial protein can provide a homology-model of a homologous human protein which may be a possible drug target. Structure-based drug design has successfully been applied to the identification of new protease inhibitors using homology models constructed from structural information of homologous enzymes having limited sequence identity (20-33%) to the inhibited enzymes (52). The structure of the bacterial protein may be very relevant for the design of a drug with the human protein as target since both proteins are likely to have a very similar active site with key conserved residues which may be the site of interaction for the drug.

All the aforementioned applications of the invention will greatly benefit from the methods presented here, wherein well-suited candidates of homologous proteins may be obtained more readily, than by prior art methods.

As an optional step of the method of the invention, additional segments may be subsequently obtained from the sample comprising the one or more target sequence or a part thereof, wherein the additional nucleic acid segment codes for the candidate protein or a part thereof. For example, if a target sequence contains a relatively short segment, such as a fragment between regions complementary to two primers, it may be preferred to obtain from the broad diversity sample complementary or more complete portions of the gene comprising the target sequence to express as a candidate protein. Selection of candidates in silico can be done using these partial gene sequences and more specific primers can then be designed for the amplification of the complete genes (53,54). Partial gene fragments can also be used in hybridization experiments to identify corresponding gene in a library of nucleic acids such as in a library of vectors containing genomic fragments (55).

The comparison of sequences which is used to direct selection of candidates can also provide information directing experimentation in other ways. This may be for example be indications of the borders of domains in multi-domain proteins which may lead to the use of gene fragments and protein fragments (e.g., single domains) in addition to or instead of full-length genes and proteins. Similarly, the possible presence of unstructured termini can be identified and eliminated in the expressed protein.

The selected target sequences and the optionally obtained additional nucleic acid segments are expressed in a suitable expression system using well known techniques of the art. Such methods include the use of a suitable recombinant expression vector comprising a nucleic acid target sequence of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably or operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in ref. (56). Regulatory sequences include those which direct constitutive expression of a nucleotide sequence in many types of host cell and those which direct expression of the nucleotide sequence only in certain host cells. It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides or genetically modified polypeptides, which constitute candidate proteins obtained by the invention. The expression system may e.g., be designed to produce a fusion protein of the desired gene product and an additional purification tag such as a His-tag or a chitin-binding domain (57). Expression may be conveniently monitored with SDS-PAGE (sodium dodecyl sulphate polyacrylamie gel electrophoresis) of whole cell lysates.

Expression of selected genes or gene fragments can conveniently be done in a suitable hosts, both prokaryotic or eukaryotic cells, e.g., bacterial cells such as Escherichia coli by cloning into an appropriate expression vector such as “ATG vectors” (58). The expression of the gene may be controlled by using a vector with a suitable promoter system such as the T7 promoter (59). Alternatively, the recombinant expression vector can be transcribed and translated in vitro, for example using T7 promoter regulatory sequences and T7 polymerase.

To further broaden the diversity available with the method of the invention, methods are disclosed wherein the nucleic acids are biologically normalized by combining different enriched microbial populations prior to extracting the nucleic acids. Samples containing microorganisms are obtained from multiple natural environments such as described above. The samples can then be enriched as described herein. The enriched microbial populations are combined, and nucleic acids extracted, isolated and characterized, thereby producing a normalized representation of the genomes derived from these multiple enriched broad diversity samples. The enriched microbial population also provides large quantities of cells allowing use of different isolation techniques that ensure little fragmentation of the DNA, such as casting the cells in agar plugs and using mild enzymatic methods of cell lysis and DNA purification in order to obtain sufficiently large fragments for construction of bacteria metagenomic libraries (60). Such libraries facilitate the genetic screening for whole genes and operons coding for enzymes involved in cooperative synthesis of low weight secondary metabolites. Thus, in certain embodiments of the invention, the plurality of nucleic acid segments is comprised of a metagenomic library.

Normalized gene libraries useful for screening may also be prepared by cultivating individual species separately and then mixing them in approximately equal proportions to each other before DNA isolation. The advantages with using cultivated species is that large amounts of un-fragmented DNA which is free from enzyme inhibitors, is more easily isolated and purified from microbes freshly cultivated than from “dirty” environmental samples that adversely affects the quality of the DNA, where the microbes are mostly dormant or in unknown physiological state. Such mixing of fresh cultures can readily be used for species that are present in strain collections or that can be easily isolated with current laboratory techniques. It is apparent that traditional laboratory isolations and cultivation of most uncultivated species would be an impossible task, the solution to this problem is achieved by the enrichment methods described herein.

In a further aspect of the invention, a method is provided for obtaining a crystallized protein comprising: obtaining a candidate protein with the method of the invention; and crystallizing said candidate protein. The candidate protein is expressed as described above, typically it is purified with suitable standard purification methods, such as e.g., liquid chromatography (61). Columns with resins specific for an affinity purification using purification tags can be used to simplify purification. A heat-denaturation step can be effectively used as a purification step for thermostable proteins expressed in a mesophilic host such as E. coli (62). Purity of protein preparations can be checked during purification with SDS-PAGE. Protein preparations can be analyzed with different techniques to evaluate their suitability for crystallization trials and to establish conditions more suitable for a particular protein. This includes circular dichroism (63) to analyze stability and folding, light scattering to analyze if the protein preparation is monodisperse (64), analytical centrifugation to analyze molecular weight distribution or mass spectrometry techniques.

Crystallization can be done by screening for appropriate conditions with suitable precipitation agents using a standard technique such as the hanging- or sitting drop vapor diffusion (65-68). Pre-made sparse matrix screens can conveniently be used for fast initial screening of many different conditions (69). Further screening for crystallization conditions and optimization can be done in a more systematic way for a particular precipitant (66). Miniaturization of crystallization experiments and robotics can be employed to automate the crystallization trials (70) in order to make it a high-throughput process. After crystals have been obtained, conditions in the presence of a cryosolvent may be found for the subsequent freezing of the crystals at cryogenic temperatures (71). Crystals can be frozen and stored using liquid nitrogen prior to data collection. Example 7 below illustrates a crystallization procedure for a specific protein.

In yet a further aspect, the invention provides a method for obtaining three-dimensional structural information of a protein from a selected protein family, comprising: obtaining a cystallized protein according to the invention as described above; collecting diffraction data for the obtained crystal of the candidate protein; optionally obtaining complementary data for phase determination of the diffraction data; and determining the protein structure by use of the obtained data.

Data collection is suitably done using a suitable x-ray source such as a laboratory x-ray generator or preferably a synchrotron x-ray source (72,73) especially for multiple wavelength experiments such as MAD (9). An example of the process of a structure determination, including the use of MAD, is outlined in Example 7. Crystal mounting and data collection using frozen crystals requires the use of cryogenic equipment installed by the laboratory generator or at the synchrotron beamline. Data can be recorded using special detectors, such as image plates or CCD (charged coupled device) detectors, and the appropriate goniostat and other equipment for the alignment and controlled movement of the crystal during data collection (74-76). The data collection process can also be automated to some extent. Image data processing can be done with software such as Denzo (77) and data reduction and general crystallographic computing can done with various programs including those in the CCP4 package (78).

Phasing may be done with any of the methods known to those skilled in the art. Phase determination in the crystallography of biological macromolecules includes SIR or MIR, with or without anomalous scattering (1,7,8) and MAD (9). These methods require the use of heavy atom derivatives of the protein which can be obtained for example by soaking of protein crystals in heavy atom compound solutions (7) or by expression of the protein in a suitable host in the presence of selenomethionine to make selenomethionine-substituted protein (79). Position of heavy atom scatterer can be found with different methods, including the use of automated programs such as SOLVE (80), refinement of heavy atom parameters and phase calculation can be done with programs such as SHARP (81) and density modification with programs such as DM (82). Phasing can also be achieved with molecular replacement if the structure of a similar homologous protein is available (83-85).

Interpretation of the electron density maps can be done through manual model building such as with the program O (86) or with more automatic procedures (87) depending on the quality of the maps. Refinement of coordinates can be done the program CNS (88). Coordinates made publicly available are normally deposited in the Protein Data Bank (89,90).

The crystallographic methods and specific software mentioned here is ment to provide examples of methods and computing tools currently in use in the art. Other methods and software known to those skilled in the art can also conviniently be used for structure determination using x-ray crystallography. It is also undertood that structure determination by other methods, such as by nuclear magnetic resonance (NMR), electron crystallography or neutron diffraction, may also benefit from the methods provided by the invention and may also be included as part of the process described.

The invention provides in yet a further aspect a method for obtaining the protein structure of a first protein from protein structure data which has insufficient phase information for a structure determination, comprising: obtaining a structure of a second protein from the same protein family with the methods according to the invention; determining the phase information for said structure data for said first protein with molecular replacement methods based on the obtained structure of said second protein; determining the protein structure by use of the initial structure data and the obtained phase information. The steps of the method are suitably performed as described herein. The structure determination steps of such an approach are illustrated in Example 7, where the structure of a human protein is obtained with the use of the structure of a closely related bacterial protein.

A yet further aspect of the invention provides method for predicting the structure of a first protein comprising: obtaining a protein structure of a second protein from the same protein family according to the invention; and predicting the structure of first second protein with homology modeling based on the structure of said first structure and of the relevant sequences.

The invention is further illustrated by the following non-limiting examples.

### EXAMPLES

### Example 1

### Oligotrophic Enrichment with Hot Spring Water in Laboratory

Samples were collected in a sulfide rich hot spring in Hveragerdi (Grensdalur), Iceland. About thirty liters of hot spring water were collected in a sterile container. Sulfur-mat or filaments were collected at 65° to 75° C. and the biomass sample was stored in a sterile flask at 4° C. All media and inoculations were prepared on the day of sampling. Three series of media with different concentration of additional supplements were prepared with 500 ml spring water as aqueous base solutions, in Erlenmeyer flasks for aerobic cultivation and in closed bottles for anaerobic processes. The following stock solutions, which had been sterilized by autoclavation were added later: 1% starch (w/v), 25% (w/v) (NH₄)₂SO₄, 12.5% NaCl (w/v) and 10% (w/v) Yeast Extract (Difco). The natural hot spring water was not autoclaved before inoculation. The biomass sample was homogenized by shaking and diluted in series with spring water down to a 10⁻⁸-fold. Each series of media (1 to 10) was inoculated with 5 ml of a specific dilution of the biomass mix. The series inoculated with 10⁻² dilution was designated as R1 to R10, the series inoculated with 10⁻⁴ was designated as G1 to G10 and the series inoculated with 10⁻⁸ as φ1 to φ10. The inoculum for the series R was specifically treated with 50 % ethanol (vol/vol) for 10 min. before inoculation. Series 2 to 6 were supplemented with 0.1% starch and 1.0% (NH₄)₂SO₄ final concentration. Series 8 to 10 with 0.002% starch and 0.02% (NH₄)₂SO₄. Series 7 with 0.02% starch and 1.0% (NH₄)₂SO₄. All series were cultivated aerobically except for series 3 and 7. Anaerobiosis was achieved by applying a vacuum to the media and saturating it with nitrogen gas (N₂). Finally, the media were reduced by adding a sterile solution of Na₂S. 9H₂O (final concentration, 0.025% [wt/vol]). Nothing was added to series 1. The pH was adjusted to 9.5 with NaOH (1 N) in series 4 and 8, and to pH 4. 0 with HCl (1 N) in series 6 and 9. In series 5 and 10, 0.5% (w/v) NaCl was added as final concentration. Media, inoculated with 10⁻⁷ dilution were prepared and supplemented with final concentration of 0.5% starch, 0.1 % and 0.01% yeast extract in spring water and designated as S, YE.1 and YE.01, respectively. All cultures were incubated at 65° C. without shaking in a incubation oven (Gallencamp).

Cells were observed with a Leica DM LB light microscope equipped with a phase-contrast oil immersion objective (magnification, ×100) and were counted by using a Petroff-Hausser chamber (depth, 0.02 mm [Hausser Scientific Partnership, Horsham Pa., USA]). Each culture was stopped when the cell concentration had reached to about 10⁷ cells/mL. Before pelleting, a 20 ml sample of each culture was removed and stored either aerobically or anaerobically at 4° C.

Results from oligotrophic enrichments in three series of natural hot spring media with different concentration of additional supplements are presented in Table 1. No growth was observed in enrichments containing 0.001% Y. E. or lower after 16 days. When 0.005% Y.E. was added after 16 days of cultivation, cell numbers in series R, G, and φ reached 10⁵-10⁸ cell/ml within 2 to 42 days.

DNA was extracted from all enrichments showing positive growth and stored at −20° C. All cultures contained Bacterial 16S rRNA genes but no Archaea 16S rRNA genes. A total of 13 enrichments were selected for creating 16S rRNA genes libraries for SSU gene sequencing (R2, R3, R6, R10, G2, G3, G5, G7, φ2, φ7, φ10 and S).

All clones were sequenced with R805 reverse primer and all sequences could be aligned to each other and to sequences in the Ribosomal database. Only sequences with reliable nucleotide sequence were edited and aligned with reference strains. Table 2 shows the closest database matches for the sequence in contigs after BLAST searches.

The results show closest matches to cultivated species that belong to seven genera (Bacillus, Thermus, Meiothermus, Caloramator, Thermoterrabacterium, Chloroflexus and Moorella), one potential new genus and five non-cultivated bacterial OTUs. One belongs to unidentified green non-sulfur bacterium clone OPB34, another to unidentified Cytophagales clone OPB88, two to new candidates for new bacterial divisions, OP9 and OP12 (97), and the last one to unidentified Thermus clone SRI248 (92).

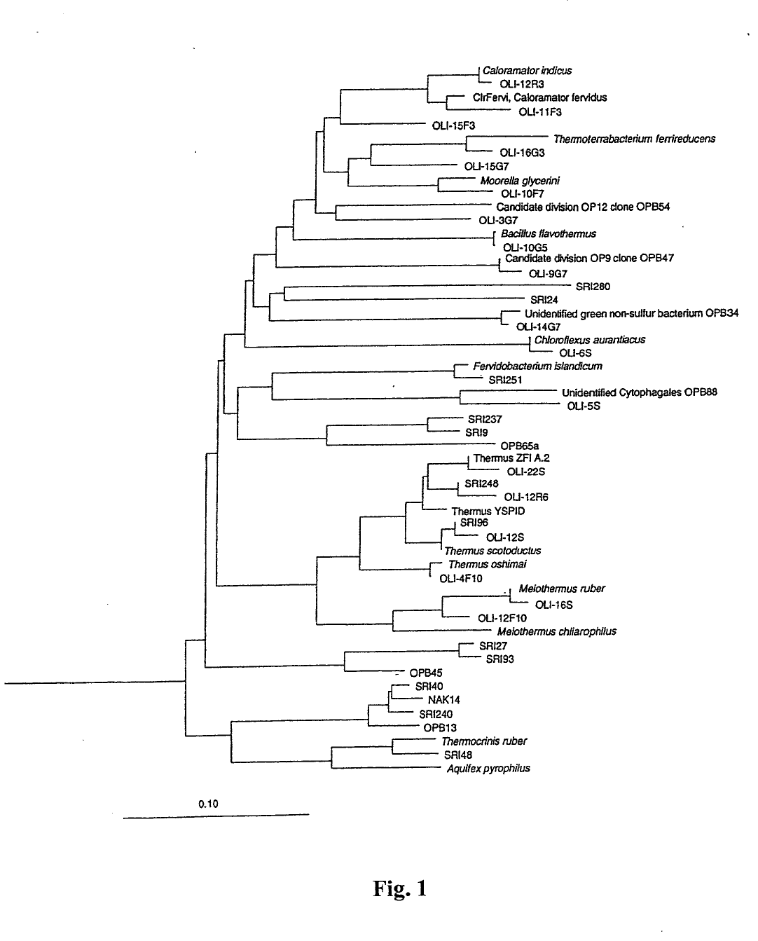

Sequence contigs from ten libraries out of thirteen selected enrichments were used for the construction of the phylogenetic tree (FIG. 1). Sequences in libraries from enrichments R2, G2 and φ2 were not used to prevent redundancy. The libraries revealed eighteen phylogenetic distinct clusters (that represent at least twelve new species in eleven genera). The oligotrophic enrichment clones were designated OLI. In enrichments G (dilution 10⁻⁴) six new species grew that were gathered to five genera. OLI-16G3 and OLI-15G7 belonged to the genus Thermoterrabacterium, although the last one was distantly related to the reference sequence. OLI-3G7 and OLI-9G7 were related to candidate division OP12 and OP9, respectively (91). OLI-10G5 is closely related to Bacillus flavothermus and OLI-14G7 to unidentified green non sulfur bacterium OPB34 (91). In enrichments R (dilution 10⁻²) two new species grew that were gathered in two genera. OLI-12R3 was closely related to Caloramator indicus and OLI-12R6 to Thermus SRI248 (92). Enrichment S (dilution 10⁻⁷) gave species belonging to five genera. Clone OLI-6S was closely related to Chloroflexus aurantiacus and clone OLI-16S to Meiothermus ruber. OLI-22S and OLI-12S belonged to Thermus ZA.2 and Thermus SRI96 respectively (92). OLI-5S was only distantly related to unidentified Cytophagales OPB88 (91). Finally, in φ enrichments (dilution 10^{-8,} clones designated F) five species were detected. OLI-11F3, OLI-10F7 and OLI-4F10 were closely related to Caloramator fervidus, Moorella glycerini and Thermus oshimae, respectively. Clone OLI-12F10 was distantly related to M. glycerini and OLI-15F3 showed very low homology to the genus Caloramator and might be a representative to a potential new genus.

The phylogenetic tree in FIG. 1 shows alignment of 16S rRNA sequences obtained with oligotrophic in situ culture method and by extracting DNA direct from environmental biomass (92). Samples were taken from the same spot. Different kind of species and genera were detected with each method. The oligotrophic method obtained much more diversity in the hot spring than the culture-independent method (92). The following known bacterial genera: Morrella, Thermoterrabacterium, Caloramator, Bacillus, Chloroflexus, Meiothermus and Thermus were detected. Other bacterial sequences belonged to non-cultivated and unidentified microorganisms, like unidentified green non-sulfur bacterium OPB34, candidate division OP12 (clone OPB54), candidate division OP9 (clone OPB47), and to unidentified Cytophagales (clone OPB88). Only Thermus was also detected with the culture-independent method.

### Example 2

### Oligotrophic Enrichment in Culture Containers in Hot Spring

Spring water from a hot spring with surface about 6 m² and 0.3 to 1.5 m deep was poured into two sterile 950 ml polyethylene containers. One of them was inoculated with 0.005% (w/v) Yeast Extract (Difco) and designated “BrusiY”, while the other one contained 0.25% (w/v) starch and designated “BrusiS”. Both BrusiY and S contained 1% (w/v) NH₄Cl (final concentration). The two containers were filled up with the spring water and then closed and placed at 1 m depth at 65° C. for 21 days. A temperature probe was used to measure the temperature inside the container with 5 minutes interval during the enrichment. Over the incubation period the temperature fluctuated between 57° C. and 72° C. The initial temperature was about 67° C., 65° C. on the second day, up again to 72° C. on the forth day, and down to 59° C. on the fifth day. After the fifth day, the temperature was fluctuating between 59° C. and 66° C. for 16 days. The fluctuations were close to being periodical with 1 or 2 days between peaks.

Both in situ oligotrophic enrichments were positive for growth. Microscopic observation showed that both contained mixed population of rod-forming and coccoid cells.

Large amounts of good quality DNA were extracted from both enrichments. Bacterial 16S rRNA genes could be amplified in both samples but no Archaea 16S rRNA genes. All clones were sequenced with R805 reverse primers and all sequences could be aligned to each other and to sequences in the ribosomal database. Only sequences with reliable nucleotide sequences were edited and aligned with reference strains. At least four genera could be detected, Thermus, Bacillus, Clostridium and Thermoanaerobacterium and at least one non-cultivated genus (Table 3).

### Example 3

### Collecting Geothermal Fluid from Hydrothermal Vents

A large quantity of hot geothermal fluid was collected from submarine hot springs, located 1.8 km offshore in the north-eastern part of the fjord Eyjafordur, Iceland. The vents occur on the east-slope, which rises from 100-m depth from the center of the fjord. At about 65 m in depth, three giant silicate cone structures, have grown at the site to heights of 33, 25 and 45 m above the sea bottom. A scuba diver was sent down with a rubber hose attached to stainless steel tube (0.4 m×10 mm). The steel tube was placed inside in a discharge opening at 27.5 m depth. Two successive 12 V booster pumps were mounted inside the tubing, few meters below the sea surface. The other end of the tube was attached to a rubber dingy. The whole system (40 m long) was rinsed with the hot fluid (around 2 L min⁻¹) for 30 min before sampling hot fluid for chemical and microbial analysis. The vent fluid was collected or concentrated directly by cross-flow filtration through sterile hollow fibre cartridges (0.22-μm filter, Amicon). The cells retained inside the cartridge (600 ml) were concentrated further in the laboratory by centrifugation. About 240 liters of 71.6° C. hot vent fluid, from a vent at 27.5 m depth was pumped and concentrated to 600 ml by filtration and pellated in an eppendorf tube.

The hydrothermal fluid had only about 0.1% contamination by seawater and was also used for oligotrophic enrichments as described in Example 1. Microscopic evaluation after 14 days in oligotrophic enrichments at 65 to 80° C. revealed complex community of cells.

DNA was successfully extracted from the concentrated biomass. Sequencing of environmental clones revealed both Bacteria (45 clones) and Korarchaea (10 clones) sequences (Table 5). The thermophilic taxonomic divisions of Bacteria represented by the clones, included mostly the order Aquificales and one unidentifed Nitrospira clone. Three clones were closest to the mesophilic divisions of Proteobacteria and Firmicutes.

### Example 4

### DNA Isolation

Cell pellets were obtained from each culture by centrifugation for 30 minutes at 8.000 rpm (Sorval) and 4° C.

Cells were disrupted with a sterile mortar (or homogenizer) and incubated for 1 hour at 37° C. in lysis TNE buffer (Tris-NaCl-EDTA, (100 mM, 100 mM, 50 mM), pH 8.0 and 1 mg/ml (final concentration) Lysozyme (Sigma), and for 2 hours at 50° C. with 1% SDS, 1% Sarcocyl and 1 mg/ml Proteinase K (Sigma, final concentrations). Gently mixed by inversion. The protein fraction was removed with several extractions with Phenol:Chloroform:Isoamyl alcohol (Sigma, 25:24:1), pH 8.0. Nucleic acids were ethanol-precipitated and dried during 10 minutes of vacuum centrifugation (SpeedVac). DNA was finally resuspended in 100 μl of TE solution (Tris-EDTA, (100 mM, 50 mM)), pH 8.0 and its quality analyzed on a 0.8% TAE-agarose gel electrophoresis. DNA was stored at −20° C.

### Example 5

### Diversity Analysis

Bacterial and Archaeal 16S ribosomal RNA genes were specifically amplified with universal oligonucleotide primer sets. The following Bacterial (Escherichia coli) primers were used:

| | | |
|---|---|---|
| Forward primer (F9) | 5′-GAGTTTGATCCTGGCTCAG-3′ | (SEQ ID NO.:1) |
| Forward primer (F515) | 5′-GTCCCAGCAGCCGCGGTAAATAC-3′ | (SEQ ID NO.:2) |
| Reverse primer (R805) | 5′-GACTACCGGGTATCTAATCC-3′ | (SEQ ID NO.:3) |
| Reverse primer (R1544) | 5′-AGAAAGGAGGTGATCCA-3′ | (SEQ ID NO.:4) |

The Archaea specific primer set used was 23 FPL and 1391R (93).

| | |
|---|---|
| Forward primer (23 FPL) | |
| 5′-GCGGATCCGCGGCCGCTGCAGAYCTGGTYGATYCTGCC-′3; | (SEQ ID NO.:5) |
| Y indicates pyrimidine substitution. | |
| Reverse primer (1391R) | |
| 5′-GACGGGCGGTGTGTRCA-3′; | (SEQ ID NO.:6) |
| R indicates purine substitution. | |

The PCR solutions were prepared as follows: 4 μl of 10× Buffer (from kit), 4 μl of dNTPs (10 mM), 1 μl of primer (20 mM) forward and reverse, 1 μl of template DNA (series of dilutions), 0.5 μl of DNA polymerase and 28.5 μl of sterile water (final volume of mix 40 μl). The PCR amplifications of Bacterial and Archaea SSU genes were performed by using DyNAzyme polymerase (Finnzyme) and with Taq DNA polymerase (QIAGEN) respectively, according to the manufactures instruction. Two protocols were used for amplification of the SSU genes (92). Bacterial 16S rRNA genes amplification reactions were performed with an initial denaturation step at 95° C. for 5 min and 85° C. for 1 min, followed by 25 amplification cycles of 95° C. for 40 sec, 42° C. for 60 sec and 72° C. for 3 min, extension was at 72° C. for 7 min. Amplifications for Archaeal SSU genes were performed with an initial denaturation step at 94° C. for 5 min then followed by 40 cycles of 94° C. for 90 sec, 55° C. for 90 sec and 72° C. for 2 min and extension at 72° C. for 7 min. These protocols were optimized experimentally by modifying number of cycles, annealing temperature, concentration of DNA and concentration of primers to obtain pure PCR product. PCR products were analyzed on a 0.8% TAE-agarose gel electrophoresis and kept at 4° C. until cloning. The amplification reactions were performed on a GeneAmp PCR System 9700 thermal cycler (PE Applied Biosystems). Libraries of fresh PCR products were constructed in E. coli cells by using the Cloning Kit (Invitrogen), according to the manufacturer. PCR products from different primer sets within enrichments were pooled before cloning.

Plasmid DNA's from single colonies were isolated with an automatic plasmid isolation apparatus (AutoGen 740 robot). The DNA was sequenced with an ABI 377 DNA sequencer by using the BigDye Terminator Cycle Sequencing kit (PE Applied Biosystems) according to the manufacturer. The SSU rRNA genes were sequenced with the reverse primer R805, 5′-GACTACCGGGTATCTAATCC-3′ (SEQ ID NO.: 3) Sequences were analyzed with the Sequencing analysis software (ABI), and sequence contigs were built up on maximum likelihood within all sequences by the software. After BLAST searches (http://www.ncbi.nih.nlm.gov/BLAST), the sequences (about 300-400 bases long) were manually aligned with closely related sequences obtained from the Ribosomal Database Project (RDP; http//rrna.uia.ac.be/rrna/ssu/forms/index) using ClustalX 1.8 software (37), and DCSE V3. 4 software (Dedicated Comparative Sequence Editor, De Rijk et al., Department of Biochemistry, University of Antwerp). SeqPup0.6 (D. C, Gilbert, Biology Dpt, Indiana University, Bloomington) was used as a file translator. Distance trees were constructed by the neighbor joining algorithms with the ARB software (Strunk et al., Lehrstuhl fir Mikrobiologie, Technical University of Munich).

### Example 6

### PCR-Amplification of Unknown Amylase Gene Sequences from Enrichments

Primers were designed according to the CODEHOP strategy by using the CODEHOP program (38). The primers were degenerate at the 3′ core region of length 11-12 bp across four codons of highly conserved amino acids. In contrast they were non-degenerate at the 5′ region (consensus clamp region) of 18-25 bp with the most probable nucleotide predicted for each position. Reducing the length of the 3′ core to a minimum decreases the total number of individual primers in the degenerate primer pool. The 5′ non-degenerate consensus clamp stabilizes hybridization of the 3′ degenerate core with the target template.

For the primer construction, amino acid sequences of various amylolytic enzymes were retrieved from protein database (94) and aligned by using CLUSTALX version 1.8. (37). Furthermore, blocks of multiply aligned amino acid sequences, established with the program Blockmaker (95) were used as input for the CODEHOP program. Subsequently, a set of forward and reverse primers were constructed, aimed to hybridize to the DNA coding sequences of the conserved A- and B-regions, of amylolytic enzymes, respectively (96).

Nucleic acids were extracted from harvested cells obtained from oligotrophic enrichments cultures in containers located in a hot spring as previously described (EXAMPLE 2). Each forward primer was tested against each reverse primer in a matrix of PCR-reactions.

The PCR amplifications were performed with 0.5 U of DyNAzyme DNA polymerase (Finnzyme), 1-10 ng of template DNA, a 0.1 μM concentration of each synthetic primer, a 0.2 mM concentration of each deoxynucleoside triphosphate and 1.5 mM MgCl₂ in the buffer recommended by the manufacturer. A total of 30 cycles were performed; each cycle consisted of denaturing at 94° C. for 50 s, annealing at 50° C. for 50 s, and extension at 72° C. for 60 s.

Cloning and sequencing of the PCR products was carried out as previously described for the SSU rRNA genes except that M13 forward and reverse primers were used for the sequencing of the cloned PCR products. All data base searches were run with the program BLASTX on server from the National Center for Biotechnology Information, Bethesda, Md., USA (34). The alignment of the derived amino acid sequences and construction of phylogenetic trees was as described for the SSU rRNA genes.

To determine the nature and extent of amylolytic enzymes within enrichment cultures, we designed primers to detect unknown amylase-family gene sequences. The amino acid sequences of 199 amylolytic enzymes were multiply aligned and classified according to the alignment. Two sequence regions (A and B) (96) separated by ˜80-200 amino acids were chosen as primer target sites. Sixteen different forward primers with region A as a target site and seven different reverse primers with region B as a target site were constructed according to the classification. The degeneracy of the primer pools ranged from 16-fold to 64-fold and they were 29-32 bp in length.

Electrophoretic analysis revealed bands of expected sizes (˜250-600 bp) in amplification reactions with certain primer combinations. The corresponding fragments were cloned and 8-12 clones from each band were sequenced. Of 35 cloned fragments, five different corresponded to amylolytic enzyme gene sequences. The results are summarized in Table 4 and FIG. 3. No sequence was observed in both types of enrichment cultures. The “BrusiY” amylase sequences revealed similarity to Thermus sequences in accordance to the rRNA sequence analysis, which detected Thermus bacteria only in BrusiY.

### Example 7

### Structure Determination of a Bacterial and a Human Member of a Protein Family

The 2-oxo acid dehydrogenase multienzyme complexes contain different enzyme components with homologous components in different types of complexes (97). In order to determine the structure of an El component of a multienzyme complex of this type, work was started with homologous El components from 4 different species and belonging to two different types of multienzyme complexes: E1p from Azetobacter vinelandii, E1p from Bacillus stearothermophilus, E1b from Pseudomonas putida and human E1b. Crystallization attempts were made with the four purified proteins in parallel in the hope that at least one would allow successful crystallization and structure determination. Initial crystallization trials were made with a variety of crystallization screens, both systematic screens with various precipitation agents, such as ammonium sulphate and polyethylene glycol (PEG) of different molecular weights, and random screens such as “Magic 96” (commercially available as “Wizard 1 and 2” from Emerald Bio structures, http://www.emeraldbiostructures.com). Promising conditions were expanded with more systematic screening and optimization.

Crystals of Pseudomonas putida E1b were obtained with phosphate as precipitation agent (98). Crystals were grown using sitting-drop vapor diffusion by mixing protein solution and precipitant solution (ratio between 1:1 and 6:1 for a total of 2-10 microliters). The protein solution contained ca. 8 mg/ml protein, 50 mM potassium phosphate pH 7.5, 1 mM Thiamine diphosphate (ThDP), 4 mM MgCl₂, 10 mM L-valine, 4-12 mM dithiothreitol (DTT) and optionally 2 mM α-chloroisocaproate (enzyme inhibitor). The precipitant solution contained 1.8-2.5 M sodium phosphate/potassium phosphate pH 5.2, 0.01% NaN₃ and 4-12 mM DTT. Crystals were frozen with liquid nitrogen in a solution containing 20-25% glycerol, 2.0-2.5 M ammonium sulphate, 1 mM Thiamine diphosphate (ThDP), 4 mM MgCl₂, 10 mM L-valine, 4-12 mM dithiothreitol (DTT) and optionally 2 mM α-chloroisocaproate. Native data were collected to 2.6 Å resolution at CHESS (Cornell High Energy Synchrotron Source, NY, U.S.A. ) beamline F1 at cryogenic temperatures. The crystals belonged to space group I4₁22 with cell-dimensions a=b=101 Å and c=382 Å. The protein was also expressed in a Pseudomonas putida methionine auxotroph with L-selenomethionine in the medium to produce a selenomethionine-substituted protein. MAD data were collected on selenomethionine protein crystals at three different wavelengths at ESRF (European Synchrotron Radiation Facility, Grenoble, France) beamline BM14. The data were processed with programs Denzo and Scalepack (77) and programs of the CCP4 suite (78). Phase information and traceable electron density map was obtained using the MAD data after location of the 22 Se atoms and refinement of the heavy atom parameters using the program SHARP (81). Interpretation of the electron density map and model building was done using the program O (86) and refinement of the atomic model with programs X-PLOR (99) and CNS (88). The results of the structure determination have been previously published (98) and the structural coordinates deposited in the Protein Data Bank (accession code 1qs0).

Extensive efforts with repeated preparations of variable constructs of human E1b and massive screening of crystallization conditions eventually produced thin needle-like crystals of the protein. Data was collected of native protein and of Selenomethionine-substituted protein. However, the data from the Se derivative crystals were not of sufficient quality to allow structure determination with the MAD method. The structure could only be determined with phase information obtained using molecular replacement techniques with the previously determined structure of Pseudomonas putida E1b. Determination of the structure of the bacterial protein was thus a prerequisite for the structure determination of the human protein (48). The structures of Pseudomonas putida E1b and the human E1b are very similar and illustrative of the high structural similarity that can exist between homologous proteins in bacteria and higher eukaryotes. The coordinates of the structure of human E1b are deposited in the Protein Data Bank (accession code 1dtw).

Large crystals of Bacillus stearothermophilus E1p could be readily obtained and data could be collected from these crystals to a resolution sufficient for structure determination. However, the crystals suffered from devious imperfection, i.e., twinning, that could only be revealed after analysis of the data. The twinning problem prevented successful structure determination.

Crystals could not be obtained of Azetobacter vinelandii E1p despite extensive screening.

This example illustrates some of the problems that can occur at different stages of the structure determination process even well beyond the crystallization step. It also shows the benefits of an approach using homologous proteins from different sources and how the structure determination of one protein can ultimately be crucial for the determination of the structure of a related protein.

TABLE 1
Results of oligotrophic enrichments done in natural fluid base. Yeast extract (0.005%
final concentration) was added to all cultures after 16 days of incubation.

| | | | | | | | Cultiv. | | |
|---|---|---|---|---|---|---|---|---|---|
| Inoculum | Enrichment | Starch | (NH₄)₂SO₄ | Head | | NaCl | time | Microscopic | |
| dilution | code | (w/v) | (w/v) | space | pH | (%) | (days) | observation | Cells/ml |
| 10⁻² | R1 | — | — | | — | | 18 | Rods | 10⁶-10⁷ |
| | R2 | 0.1% | 1.0% | air | — | | 21 | Rods | 10⁶-10⁷ |
| | R3 | 0.1% | 1.0% | N₂ | — | | 22 | Long & thin rods | N.D. |
| | R4 | 0.1% | 1.0% | air | 9.5 | | 18 | Rods | 10⁵-10⁶ |
| | R5 | 0.1% | 1.0% | air | — | 0.5 | 18 | Rods | N.D. |
| | R6 | 0.1% | 1.0% | air | 4 | | 18 | Rods | 10⁶ |
| | R7 | 0.002% | 1.0% | N₂ | — | | 22 | Cocci, long & thin rods | 10⁶-10⁷ |
| | R8 | 0.002% | 0.02% | air | 9.5 | | 18 | Small rods | 10⁶-10⁷ |
| | R9 | 0.002% | 0.02% | air | 4 | | 18 | Rods of all sizes | 10⁶ |
| | R10 | 0.002% | 0.02% | air | — | 0.5 | 60 | Rods & spores | 10⁶-10⁷ |
| 10⁻⁴ | G1 | — | — | air | — | | 21 | Rods of all size, | 10⁵-10⁶ |
| | | | | | | | | filaments | |
| | G2 | 0.1% | 1.0% | air | — | | 18 | Very thin & small rods | 10⁶-10⁷ |
| | G3 | 0.1% | 1.0% | N₂ | — | | 22 | Small & thin rods | 10⁵-10⁶ |
| | G4 | 0.1% | 1.0% | air | 9.5 | | 18 | Thin & small rods | >10⁷ |
| | G5 | 0.2% | 1.0% | air | — | 0.5 | 18 | Rods | 10⁶-10⁷ |
| | G6 | 0.1% | 1.0% | air | 4 | | 74 | No biomass | N.D. |
| | G7 | 0.002% | 1.0% | N₂ | — | | 50 | Cocci & spores, rods | 10⁶-10⁷ |
| | Φ9 | 0.002% | 0.02% | air | 4 | | 21 | Very small & thin rods | 10⁶ |
| | Φ10 | 0.002% | 0.02% | air | — | 0.5 | 60 | Rods & spores | 10⁷-10⁸ |
| 10⁻⁷ | YE.1 | — | | air | — | 0.1 | 7 | Rods of all size | 10⁶-10⁷ |
| | YE.01 | — | | air | — | 0.01 | 11 | Rods of all size | 10⁶-10⁷ |
| | S | 0.5% | — | air | — | | 12 | Rods | 10⁶-10⁷ |

TABLE 2
Identification of cloned 16S rRNA sequences (320 clones from 13
enrichments) from oligiotrophic enrichments based on Ribosomal Database BLAST
searches.

| Clones | No. of | | |
|---|---|---|---|
| Code | clones | Bacterial division | Closest Database Match (%) |
| OLI-R2 | 38 | Thermus-Deinococcus group | Thermus SRI96 (99%) |
| | 11 | Thermus-Deinococcus group | Thermus oshimai (99%) |
| | 1 | low G + C gram positives | Bacillus flavothermus (99%) |
| OLI-R3 | 7 | low G + C gram positives | Caloramator fervidus (90%) |
| | 1 | low G + C gram positives | Caloramator indicus (99%) |
| OLI-R6 | 16 | Thermus-Deinococcus group | T. SRI96 (99%) |
| | 1 | Thermus-Deinococcus group | Thermus SRI248 (98%) |
| OLI-R10 | 11 | Thermus-Deinococcus group | T. oshimai (99%) |
| OLI-G2 | 25 | low G + C gram positives | B. flavothermus (99%) |
| | 18 | Thermus-Deinococcus group | T. SRI96 (99%) |
| OLI-G3^{a} | 17 | low G + C gram positives | B. flavothermus (99%) |
| | 3 | low G + C gram positives | Thermoterrabacterium ferrireducens |
| | | | (93%) |
| OLI-G3^{b} | 2 | low G + C gram positives | C. fervidus (99%) |
| | 2 | low G + C gram positives | B. flavothermus (99%) |
| OLI-G5 | 16 | low G + C gram positives | B. flavothermus (99%) |
| OLI-G7 | 8 | New division candidate | Candidate OP9 clone OPB47 (99%) |
| | 7 | Green non-sulfur bacteria | Unidentified green non-sulfur bacterium |
| | | | clone OPB34 (100%) |
| | 4 | low G + C gram positives | Moorella glycerini (96%) |
| | 3 | low G + C gram positives | Thermoterrabacterium ferrireducens |
| | | | (93%) |
| | 2 | New division candidate | Candidate OP12 clone OPB54 (91%) |
| OLI-φ2 | 46 | Thermus-Deinococcus group | T. SRI96 (99%) |
| | 2 | Thermus-Deinococcus group | T. oshimai (99%) |
| | 6 | low G + C gram positives | B. flavothermus (99%) |
| OLI-φ3 | 7 | low G + C gram positives | C. fervidus (99%) |
| | 5 | low G + C gram positives | C. fervidus (99%) |
| | 3 | low G + C gram positives | B. flavothermus (99%) |
| OLI-φ7 | 7 | Green non-sulfur bacteria | Unidentified green non-sulfur bacterium |
| | | | clone OPB34 (100%) |
| | 6 | low G + C gram positives | C. fervidus (99%) |
| | 5 | low G + C gram positives | M. glycerini (96%) |
| OLI-φ10 | 10 | Thermus-Deinococcus group | M. ruber (94%) |
| | 9 | Thermus-Deinococcus group | T. oshimai (99%) |
| OLI-S | 13 | Thermus-Deinococcus group | M. ruber (99%) |
| | 3 | Green non-sulfur bacteria | Chloroflexus aurantiacus (98%) |
| | 3 | Thermus-Deinococcus group | T. SRI96 (99%) |
| | 1 | Thermus-Deinococcus group | Thermus ZF A.2 (98%) |
| | 1 | Bacteriodes-Cytophaga- | Unidentified Cytophagales |
| | | Flexibacter | clone OPB88 (89%) |

TABLE 3
Identification of SSU rRNA sequences derived from
Bacterial libraries obtained from In situ oligiotrophic
enrichments BrusiY and BrusiS placed in the hot spring.

| In situ oligiotrophic enrichment | | In situ oligiotrophic enrichment | |
|---|---|---|---|
| BrusiY | | BrusiS | |
| | Closest | Closest | Closest |
| Closest Species | database | Species | database |
| Representative | match (%) | Representative | match (%) |
| Clostridium sp. | 84-94 | Clostridium sp. | 95-97 |
| Clostridium sp. | 98 | Clostridium sp. | 99 |
| Alicyclobacillus | 99 | Alicyclobacillus | 87-99 |
| Thermus | 88-100 | Thermoanaerobacter finii | 95 |
| antranikianus | | | |
| Unidentified | 84 | | 97 |
| | | | 90 |
| | | | 88 |
| Total Clones 69 | | Total Clones 62 | |

TABLE 4
Amylases and related enzymes from in situ
oligotrophic enrichment cultures.

| Clone | Amylase | | PCR primers | |
|---|---|---|---|---|
| code | signature | origin | (f/r) | Homologous enzyme |
| | | | | Enzyme |
| | | | | Bacteria |
| | | | | Amino acid |
| | | | | sequence identity |
| 2.26 | am1 | BrusiS | 15.Equ-FNH-f | Cyclomaltodextrinase |
| | | | 26.Equ-GWR-r | Alicyclobacillus |
| | | | | acidocaldarius |
| | | | | 86% |
| 2.27 | am2 | BrusiS | 5.Bac-VNH-f | α-amylase |
| | | | 31.Equ-AKH-r | Alicyclobacillus |
| | | | | acidocaldarius |
| | | | | 91% |
| 14.1 | am3 | BrusiY | 15.Equ-FNH-f | glycosyl hydrolase |
| | | | 26.Equ-GWR-re | Deinococcus |
| | | | | radiodurans |
| | | | | 59% |
| 14.2 | am4 | BrusiY | 15.Equ-FNH-f | glycosyl hydrolase |
| | | | 26.Equ-GWR-r | Deinococcus |
| | | | | radiodurans |
| | | | | 57% |
| 1.7 | am5 | BrusiY | 16.Equ-YNH-f | α-glucosidase |
| | | | 25.Equ-GFR-r | Thermus aquatic |
| | | | | 81% |

TABLE 5
Molecular diversity analysis of environmental
DNA in geothermal fluid from hydrothermal vent.

| Type | No. of | | |
|---|---|---|---|
| sequence | clones | Bacterial division | Closest database match (%) |
| OTU | | | |
| Bacteria | | | |
| library | | | |
| ST22 | 1 | Nitrospira group | Unidentified |
| | | | (OPB67A 97%) |
| ST56 | 15 | Aquificales | Hydrogenobacter |
| | | | thermophilus TK-6 |
| | | | (90%) |
| ST10 | 26 | Aquificales | EM17 (97%) |
| ST43 | 1 | Firmicutes | Propionobacterium |
| | | | acnes (96%) |
| ST12 | 1 | α-Proteobacteria | Caulobacter |
| | | | crescentus (99%) |
| ST50 | 1 | β-Proteobacteria | Alcaligenes |
| | | | sp. (99%) |
| Archaea library | | | |
| ST89 | 10 | Korarchaeota | Clone pJP78 (99%) |

References Cited

1. Hol, Nat. Struct. Biol. 7:964-966 (2000)

2. Blundell & Johnson, Protein Crystallography Academic Press, London (1976)

3. Drenth, Principles of X-ray Crystallography of Proteins (Springer Verlag, New York, 1994)

4. Hendrickson, Trends Biochem Sci 25:637-643 (2000)

5. Burley, Nat. Struct. Biol. 7:932-934 (2000)

6. Cassetta et al., J. Syncr. Radiation 6: 822-833 (1999)

7. Isomorphous Replacement and Anomalous scattering (Eds. Wolf et al., Science and Engineering Council, Warrington, WA44AD, UK (1991)

8. Ke, Methods Enzymol. 276:448-461 (1997)

9. Hendrickson, Science 254:51-8 (1991)

10. Terwilliger et al., Protein Sci. 7:1851-1856 (1998)

11. Rost, Structure 6: 259-263 (1998)

12. Brenner, Nat. Struct. Biol. 7:967-969 (2000)

13. Terwilliger, Nat. Struct. Biol. 7:935-939 (2000)

14. Hwang et al., Nat. Struct. Biol. 6:691-696 (1999)

15. Yokoyama et al., Nat. Struct. Biol. 7:943-945 (2000)

16. Saitou, N., and M. Nei, Mol. Biol. Evol. 4: 406-425 (1987)

17. Alexander, Extreme environments. Mechanisms of microbial adaptation. Ed. Heinrich, New York Academic Press, 3-25 (1976)

18. Stevenson, Microbiol Sci 2:367-368 (1985)

19. U.S. Pat. No. 6,001,574

20. Roszak & Colwell, Microbiol. Rev. 51: 365-379 (1987)

21. Stanley & Konopka, Annu. Rev. Microbiol. 39: 321-346 (1985)

22. Sambrook & Maniatis, Molecular cloning: a laboratory manual, 2nd ed., (Cold Spring Harbour Laboratory Press, 1989)

23. Jackson et al., Appl. Environm. Microbiol. 63:4992-4995 (1997)

24. Miller et al., Appl. Environm. Microbiol. 65: 4715-4724 (1999)

25. PCR Technology: Principles and Applications for DNA Amplification (Ed. H. A. Erlich, Freeman Press, New York, N.Y., 1992)

26. PCR Protocols: A Guide to Methods and Applications (Eds. Innis, et al., Academic Press, San Diego, Calif., 1990)

27. Mattila et al., Nucleic Acids Res., 19:4967 (1991);

28. Eckert et al., PCR Methods and Applications, 1:17 (1991)

29. PCR (Eds. McPherson et al., IRL Press, Oxford)

30. U.S. Pat. No. 4,683,202

31. Morris, D. D. et al., Appl. Environ. Microbiol. 61:2262-2269 (1995)

32. Shyamala, V. & Ames, G. F., Gene. 84:1-8 (1989)

33. Timothy, M. R., et al., Nucleic Acids Research 2:1628-1635 (1998)

34. Altschul et al., Nucleic Acids Res. 25:3389-3402 (1997)

35. Bateman et al., Nucl. Acids Res. 28:263-266 (2000)

36. Tatusov et al., Nucleic Acids Res. 29:22-28 (2001)

37. Thompson et al., Nucleic Acid Res. 22:4673-4680 (1994)

38. Rose et al., Nucleic Acids Res. 26:1628-35. (1998)

39. Sanger, Proc Natl. Acad. Sci. USA 74:5463-5467 (1977)

40. Christendat et al., Nature Struct. Biol. 7:903-909 (2000)

41. Vaghjiani et al., Biocatalysis and Biotransformation 18: 151-75 (2000)

42. Cothia & Lesk, EMBO J. 5:823-826 (1986)

43. Auerbach et al., Structure 6:769-781 (1998)

44. Macedo-Ribeiro et al., Structure 4:1291-1301 (1996)

45. Shapiro & Harris, Curr. Opin. Biotechnol. 11:31-35 (2000)

46. Skolnick et al., Nat. Biotechnol. 18:283-287 (2000)

47. Zarembinski et al., Proc. Natl. Acad. Sci. USA 95:15189-15193 (1998)

48. Aevarsson et al., Structure 8:277-291 (2000)

49. Practical Applications of Computer-Aided Drug Design (Ed. Charifson, Marcel Dekker Inc. NY, 1997)

50. Kuntz, Science 257:1078-1082 (1992)

51. Verlinde and Hol, Structure 2:577-587 (1994)

52. Ring et al., Proc. Natl. Acad. Sci. USA. 90:3583-3587 (1993)

53. Padegimas & Reichert, Anal. Biochem. 260:149-153 (1998)

54. Rudenko et al., Plant Mol. Biol., 21:723-728 (1993)

55. Heyer & Wendenburg, Appl. Environ. Microbiol 67:363-370 (2001)

56. Goeddel, Gene Expression Technology: Methods in Enzymology 185, Academic Press, San Diego, Calif. (1990)

57. Sheibani, Prep Biochem Biotechnol 29:77-90 (1999)

58. Aman & Brosius, Gene 40:183-190 (1985)

59. Studier et al., Methods Enzymol. 185:60-89 (1990)

60. Rondon, M. R. et al., Appl. Environ. Bacteriol 66: 2541-2547 (2000)

61. Scopes, Protein Purification: principles and practice (Springer Verlag, N.Y., 1994)

62. Martemyanov et al., Protein Expr. Purif. 18:257-261 (2000)

63. Price, Biotechnol. Appl. Biochem. 31:29-40 (2000)

64. Frerre-D'Amare & Burley, Structure 2:357-359 (1994)

65. Methods in Enzymology 114, Diffraction Methods of Biological Macromolecules (Eds. Wyckoff et al., Academic Press, Orlando, Fla. 1985)

66. McPherson, Crystallization of Biological Macromolecules (Cold Spring Harbor Laboratory Press, Cold Spring Harbor, N.Y., 1999)

67. Methods in Enzymology 276, Diffraction Methods of Biological Macromolecules (Eds. Carter & Sweet, Academic Press, NY, 1997) (Eds. Carter & Sweet, Academic Press, NY, 1997)

68. McPherson, Eur. J. Biochem. 189:1-23 (1990)

69. Jancarik & Kim, J. Applied Crystallog. 24:409-411 (1991)

70. Shaw Stewart & Baldock, J. Crystal Growth 196:665-673 (1999)

71. Watenpaugh, Curr. Opin. Struct. Biol. 1:1012-1015 (1991)

72. Ealick & Walter, Curr. Opin. Struct. Biol. 3:725-736 (1993)

73. Helliwell, Methods Enzymol. 276:203-217 (1997)

74. Walter et al., Structure 3:835-844 (1995)

75. Arndt, J. Appl. Crystallogr. 19:145-163 (1986)

76. Data Collection and Processing (Eds. Sawyer et al., Science and Engineering Council, Warrington, WA44AD, UK (1991)

77. Otwinowski & Minor, Methods Enzymol. 277:307-326 (1997)

78. Collaborative Computational Project Number 4, Acta Crystallogr. D 50: 760-763 (1994)

79. Hendrickson et al., EMBO J. 9:1665-1672 (1990)

80. Terwilliger & Berendzen, Acta Crystallogr. 55:849-861 (1999)

81. De La Fortelle & Bricogne, Methods Enzymol. 276:472-494 (1997)

82. Cowtan, Joint CCP4 and ESF-EACBM Newsletter on Protein Crystallography 31:34-38 (1994)

83. The Molecular Replacement Method (Ed. Rossman, Gordon & Breach, New York, 1972)

84. Fitzgerald, J. Appl. Crystallogr. 21:273-278 (1988)

85. Navazza, Acta Crystallogr. A 50:157-163 (1994)

86. Jones et al., Acta Crystallogr. A 47:110-119 (1991)

87. Perrakis et al., Nat. Struct. Biol. 6:458-463 (1999)

88. Brunger et al., Acta Crystallogr. D 54:905-921 (1998)

89. Keller et al., Acta Crystallogr. D 54:1105-1108 (1998)

90. Berman et al., Nat. Struct. Biol. 7:957-959 (2000)

91. Hugenholtz, P., C. et al., “Novel division level bacterial diversity in a Yellowstone hot spring,” J. Bacteriol. 180: 366-376 (1998)

92. Skirnisdóttir et al., Appl. Environ. Microbiol. 66:2835-2841 (2000)

93. Barns, S. M. et al., Proc. Natl. Acad. Sci. USA. 91:1609-1613 (1994)

94. Bateman, A. et al., Nucleic Acids Research 27: 260-262 (1999)

95. Henikoff, S., et al., Gene 163:17-26 (1995

96. Takehiko, Y., “Enzyme chemistry and molecular biology of amylases and related enzymes,” The amylase research society of Japan, CRC Press, pp. 81-100 (1994)

97. Reed, L. J. Multienzyme complexes. Accounts Chem. Res. 7:40-46 (1974)

98. Aevarsson A., Seger K., Turley S., Sokatch J. R., Hol W. G. J. Crystal structure of 2-oxoisovalerate and dehydrogenase and the architecture of 2-oxo acid dehydrogenase multienzyme complexes, Nat. Struct. Biol. 6:785-92 (1999)

99. Brunger, A. T., Krukowski, A. & Erickson, J. W. Slow-cooling protocols for crystallographic refinement by simulated annealing, Acta Crystallogr. A 46:585-593 (1990)

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for obtaining one or more candidate proteins for crystallization from a broad diversity sample, wherein the candidate proteins have desired characteristics to facilitate crystallization, the method comprising:
a) obtaining a broad diversity sample comprising microorganisms potentially having genes coding for one or more proteins having desired characteristics that facilitate crystallization;
b) isolating nucleic acids from the sample;
c) sequencing a plurality of nucleic acid segments comprised in the isolated nucleic acids;
d) selecting from the obtained nucleic acid sequences one or more target sequences based on suitable selection criteria;
e) optionally obtaining from the broad diversity sample one or more additional nucleic acid segments comprising the one or more target sequences or a part thereof, wherein the additional nucleic acid segment codes for the candidate protein or a part thereof;
f) expressing said one or more target sequences and/or additional nucleic acid segments; and
g) isolating expressed gene product(s) to obtain one or more candidate proteins that have characteristics that facilitate crystallization.

2. The method of claim 1, wherein the candidate proteins have desired characteristics to facilitate the process of structure determination.

3. The method of claim 1, wherein the suitable selection criteria comprise one or more criterion selected from the group consisting of:
a) a predetermined maximum hydrophobicity of any given region of a predetermined length of the sequence;
b) a predetermined minimum percentage of one or more predetermined amino acid residues;
c) a predetermined maximum percentage of one or more amino acid residues;
and combinations thereof.

4. The method of claim 3, wherein the suitable selection criteria comprise a criterion of a preselected minimum percentage of one or more amino acid residues selected from the group consisting of Asn, Gln, Glu, Asp, His, Lys and combinations thereof.

5. The method of claim 3, wherein the suitable selection criteria comprise a criterion of a preselected maximum percentage of one or more amino acid residues selected from the group consisting of Phe, Tyr, Trp and combinations thereof.

6. The method of claim 1, wherein the plurality of nucleic acid segments is selected such that it comprises nucleic acid segments suspected of coding for a protein or part of a protein of interest.

7. The method of claim 6, wherein oligonucleotide primers, derived from known sequences coding for a proteins from the selected protein family of interest, are used in sequence-based screening methods using polymerase chain reaction (PCR) to select the plurality of nucleic acid segments

8. The method of claim 1, wherein the plurality of nucleic acid segments is comprised of a metagenomic gene library.

9. The method of claim 1, wherein the one or more candidate protein is a thermostable protein.

10. The method of claim 1, wherein the obtained sample comprises microorganisms selected from the group consisting of: viruses, prokaryotic microorganisms, lower eukaryotic microorganisms and combinations thereof.

11. The method of claim 1, wherein the broad diversity sample is obtained from isolated strains of microorganisms.

12. The method of claim 11, wherein the microorganisms are thermophilic organisms.

13. The method of claim 1, wherein the broad diversity sample is obtained from a natural environment.

14. The method of claim 13, wherein the environment is a geothermal environment.

15. The method according to claim 13, wherein the broad diversity sample is enriched for a microbial population, prior to isolating nucleic acids, by
a) maintaining the sample under conditions substantially similar to the environment from which the sample was obtained to thereby expand the microbial population; and
b) allowing a sufficient quantity of a microbial population to expand;
whereby the population has been enriched.

16. The method of claim 15, wherein the nucleic acids are biologically normalized by combining different enriched microbial populations prior to extracting the nucleic acids.

17. The method of claim 7, wherein the primers are designed to preferentially screen and amplify candidate sequences from the protein family of interest that have one or more selected features.

18. The method of claim 2, wherein the suitable selection criteria benefit structure determination.

19. The method of claim 18, wherein the suitable selection criteria comprise a criterion of a desired number or ratio of a pre-determined amino acid residue.

20. The method of claim 19, wherein said criterion is a desired ratio of methionine residues.

21. The method of claim 6, wherein the candidate protein comprises an active site of a protein family.

22. The method of claim 6, wherein the protein family comprises a protein in a pathogenic organism.

23. The method of claim 6, wherein the protein family comprises a mammalian protein, including a human protein, with unknown structure.

24. The method of claim 23, wherein the mammalian protein with unknown structure is linked to a disease.

25. A method for obtaining a crystallized protein, comprising:
a) obtaining a candidate protein using the method of claim 1; and
b) crystallizing said candidate protein.

26. A method for obtaining a three-dimensional structural information of a protein from a selected protein family, comprising
a) obtaining a crystallized protein according to claim 25;
b) collecting diffraction data for the obtained crystal of the candidate protein;
c) optionally obtaining complementary data for phase determination of the diffraction data; and
d) determining the protein structure by use of the obtained data.

27. The method according to claim 26, wherein the protein structural information is used to facilitate protein design.

28. The method of claim 27, wherein the obtained plurality of nucleic acid sequences allows the determination of important functional determinants for designing proteins of new and/or improved functionality according to selected criteria.

29. The method of claim 28, where the new and/or improved functionality is achieved by rational design.

30. The method of claim 28, wherein the new and/or improved functionality is achieved by methods of directed evolution focusing on important amino acids or protein regions of importance for desired properties.

31. The method according to 26 claim wherein the structure information facilitates the design of a drug compound for combating a pathogenic organism.

32. The method according to claim 26, wherein the structure information facilitates the design of a therapeutic compound.

33. The method of claim 26, wherein selenomethionine is incorporated in the candidate protein.

34. The method according to claim 26, wherein the structural information becomes part of a database comprising structural information.

35. The method according to claim 26, wherein the structural information is used for structure prediction of proteins.

36. A method for obtaining the protein structure of a first protein from protein structure data which has insufficient phase information for a structure determination, comprising:
a) obtaining a protein structure of a second protein from the same protein family with the method of claim 26;
b) determining the phase information for said structure data for said first protein with molecular replacement methods based on the obtained structure of said second protein; and
c) determining the protein structure by use of the initial structure data and the obtained phase information.

37. A method for predicting the structure of a first protein, comprising:
a) obtaining a protein structure of a second protein from the same protein family with the method of claim 26; and
b) predicting the structure of said first protein with homology modeling based on the structure of said first protein.


SEQUENCE LISTING (6 sequences)

SEQ ID NO: 1 - DNA, 19 bp, Artificial Sequence
  bacterial primer
gagtttgatcctggctcag

SEQ ID NO: 2 - DNA, 23 bp, Artificial Sequence
  bacterial primer
gtcccagcagccgcggtaaatac

SEQ ID NO: 3 - DNA, 20 bp, Artificial Sequence
  bacterial primer
gactaccgggtatctaatcc

SEQ ID NO: 4 - DNA, 17 bp, Artificial Sequence
  bacterial primer
agaaaggaggtgatcca

SEQ ID NO: 5 - DNA, 38 bp, Artificial Sequence
  archaea primer
gcggatccgcggccgctgcagayctggtygatyctgcc

SEQ ID NO: 6 - DNA, 17 bp, Artificial Sequence
  archaea primer
gacgggcggtgtgtrca